(19) United States
(12) Patent Application Publication
(10) Pub. No.: US 2007/0116663 A1
(43) Pub. Date: May 24, 2007

(54) Composition containing polyglycerol/medium-chain fatty acid ester

(76) Inventors: Tetsuro Iwanaga, Yokkaichi-shi (JP); Kazuhito Uchida, Yokkaichi-shi (JP); Nobuyuki Takeuchi, Yokohama-shi (JP); Yoshihisa Abe, Yokohama-shi (JP)

Correspondence Address: BIRCH STEWART KOLASCH & BIRCH, PO BOX 747, FALLS CHURCH, VA, 22040-0747, US

(21) Appl. No.: 10/581,285

(22) PCT Filed: Nov. 30, 2004
(86) PCT No.: PCT/JP04/17787

§ 371 Date: Jun. 1, 2006

(30) Foreign Application Priority Data
Dec. 4, 2003 (JP) 2003-405875

(51) Int. Cl. A61K 8/37

(52) U.S. Cl. 424/70.31

(57) ABSTRACT

An object of the present invention is to provide a polyglycerol medium-chain fatty acid ester-containing composition capable of forming a water-in-oil microemulsion, the microemulsion being capable of solubilizing a large amount of water, and the composition having excellent dispersibility and self-emulsifiability in water, and cosmetics comprising the composition. The present invention relates to a polyglycerol medium-chain fatty acid ester-containing composition comprising a polyglycerol medium-chain fatty acid ester formed by esterification of a medium-chain fatty acid having 6 to 10 carbon atoms and a polyglycerol having an average degree of polymerization of 3 or more and less than 100, and a nonionic surfactant; and cosmetics comprising the polyglycerol medium-chain fatty acid ester-containing composition as defined above.

### TECHNICAL FIELD

The present invention relates to a polyglycerol medium-chain fatty acid ester-containing composition which can be suitably used for cosmetics such as cleansing cosmetics and bathing cosmetics, and cosmetics comprising the composition.

### BACKGROUND ART

In the field of cosmetics, cleansing cosmetics for the purpose of removing makeup are in the form of cream, milky lotion, liquid or the like. Also, the cleansing cosmetics can be classified by types into emulsion type, oil type and aqueous type, and have been utilized depending upon their respective uses. However, the aqueous type cleansing cosmetics containing no oil at all or a small amount of oil have disadvantage that the remover capability is weakened even though they give reduced oily feel after cleansing. Therefore, in recent years, the mainstream of the cleansing cosmetics have been a liquid oil-based makeup remover which has excellent affinity to makeup soil and is capable of easily washing off with water.

Makeup soil is removed using the makeup remover by first allowing a makeup remover to have affinity with makeup soil, thereby migrating the soil into an oily component, thereafter contacting the oily component with water to thereby form an oil-in-water emulsion of the soil-containing oily component, and directly washing off the emulsion. However, since the makeup remover is self-emulsifiable oil solution comprising a mixed system of an oily component and a surfactant, or a reversed micellar oil solution containing a small amount of water and the oil solution cannot solubilize a large amount of water has a small amount of water solubilized, phase separation take place when the amount of water exceeds the limit of solubilization, thereby forming white turbid emulsion. Therefore, an emulsion is formed spontaneously by the contact of water, so that remover capability or feel of use is worsened in a state where the skin is wet, so that its use in a bathroom is especially unsuitable.

Bathing cosmetics have been well used during bathing for the purpose of preventing rough skin, skin cracks, skin chap, improving skin conditions, or the like. The form of the bathing cosmetics includes bathing salts, bathing oils, crude medicines and the like. Among them, the self-emulsifiable bathing oil-type cosmetics comprising a mixture of an oily component and a surfactant is self-emulsified in hot water bath, and the oily component homogeneously disperses as fine oily component particles in the bath. The self-emulsifiable bathing oil-type cosmetics can be expected have various improving effects inherently owned by the oily component in emollient property, moisturizing property, anti-inflammatory disease, warm bath or the like.

However, the conventional self-emulsifiable oily bathing cosmetics comprising the mixture of an oily component and a surfactant have a insufficient dispersibility in the hot water bath. Therefore, if the bathing cosmetics are suspended in their forms without self-emulsification, not only an unpleasant experience in bathroom or feel of bathed skin such as skin moisture would be worsened, but also the bathing cosmetics are undesirably deposited on the walls of a bathtub.

The microemulsion is a system in which an oil (or water) is solubilized in an amount larger than a usual micellar solution (or reverse micellar oil solution), and its external appearance is an isotropic solution showing transparent to bluish color. In the microemulsion, there are oil-in-water type (O/W) and water-in-oil type (W/O) in the same manner as the usual macroemulsion. Furthermore, there is a bicontinuous-type microemulsion in which both an oil phase and a water phase are continuous in which large amounts of water and oil are solubilized.

It has been known that the microemulsion is obtained by adding a medium-chain alcohol having 5 to 10 carbon atoms to three components of water/surfactant/oil (see Patent Publication 1). However, the medium-chain alcohol as described above is difficult to be utilized in the use of cosmetics and medicaments from the viewpoint of skin irritation or the like.
Non-Patent Publication 1: J. H. Schulman, W. Stoeckenius, L. M. Prince, J. Phys. Chem., 63, 1677 (1959)

### DISCLOSURE OF THE INVENTION

Problems to be Solved by the Invention

The present inventors have found that a composition comprising a specified polyglycerol medium-chain fatty acid ester as a co-surfactant in addition to a nonionic surfactant forms a microemulsion capable of solubilizing a large amount of water, and has excellent water dispersiblity and self-emulsifiability in a large amount of water. The present invention has been accomplished thereby.

An object of the present invention is to provide a polyglycerol medium-chain fatty acid ester-containing composition capable of forming a water-in-oil microemulsion, the microemulsion being capable of solubilizing a large amount of water, and the composition having excellent dispersibility and self-emulsifiability in water, and cosmetics comprising the composition.

Means to Solve the Problems

The present invention relates to a polyglycerol medium-chain fatty acid ester-containing composition comprising a polyglycerol medium-chain fatty acid ester formed by esterification of a medium-chain fatty acid having 6 to 10 carbon atoms and a polyglycerol having an average degree of polymerization of 3 or more and less than 100, and a nonionic surfactant; and cosmetics comprising the polyglycerol medium-chain fatty acid ester-containing composition as defined above.

### EFFECTS OF THE INVENTION

The polyglycerol medium-chain fatty acid ester-containing composition of the present invention is capable of forming a water-in-oil microemulsion, the microemulsion being capable of solubilizing a large amount of water, and the composition having excellent dispersibility and self-emulsifiability in water. Therefore, the cosmetics comprising the composition of the present invention, for instance, cleansing cosmetics, are more likely to have affinity to makeup soil and their remover capability is not impaired even when the skin is wet, so that the cosmetics are also excellent in cleansing ability. Also, as to the bathing cosmetics, the cosmetics have excellent dispersibility in hot water bath and also excellent self-emulsifiability, so that an even higher bathing effect can be obtained.

### BEST MODE FOR CARRYING OUT THE INVENTION

The polyglycerol medium-chain fatty acid ester-containing composition of the present invention comprises at least a polyglycerol medium-chain fatty acid ester formed by esterification of a medium-chain fatty acid having a specified number of carbon atoms and a polyglycerol having a specified average degree of polymerization, and a nonionic surfactant.

The medium-chain fatty acid has a number of carbon atoms of from 6 to 10, preferably from 8 to 10, from the viewpoint of low-temperature stability. Therefore, specific examples of preferred medium-chain fatty acids include caprylic acid, capric acid and the like.

The polyglycerol has an average degree of polymerization of from 3 or more and less than 100, preferably from 3 to 50, from the viewpoint of usefulness.

The content of the cyclic compound in the polyglycerol is preferably 25% or less, more preferably 20% or less. The content of the cyclic compound can be analyzed with liquid chromatography-mass spectrometer (LC/MS).

The polyglycerol medium-chain fatty acid ester has an HLB of preferably from 7 to 15, more preferably from 8 to 13, from the viewpoint of compatibility with an oil agent and dispersibility in water. Here, HLB number is calculated by the formula:
HLB=20 (1−S/A)
wherein S is a saponification value of the ester, and A is a neutralization value of the fatty acid.

The polyglycerol medium-chain fatty acid ester shows a transparent liquid state or a pasty state, and is not solidified even at a low temperature (0° C.).

The polyglycerol medium-chain fatty acid ester can be easily obtained by esterification of a medium-chain fatty acid and a polyglycerol by a conventional method.

The polyglycerol medium-chain fatty acid ester may be used alone, or as a mixture of two or more kinds thereof having different degrees of polymerization of the polyglycerol or different degrees of esterification.

The content of the polyglycerol medium-chain fatty acid ester, for instance, in the case where the polyglycerol medium-chain fatty acid ester is used for cleansing cosmetics, is preferably from 0.1 to 80% by weight, more preferably from 1 to 50% by weight, especially preferably from 2 to 30% of the polyglycerol medium-chain fatty acid ester-containing composition, from the viewpoint of affinity to makeup soil.

The nonionic surfactant includes polyoxyethylene alkyl ethers, glycerol fatty acid esters, polyglycerol fatty acid esters (except for the above-mentioned polyglycerol medium-chain fatty acid ester), polyalkylene glycol fatty acid esters, sorbitan fatty acid esters, sugar fatty acid esters, pentaerythritol fatty acid esters, fatty acid alkanolamides, ethers formed between a polyoxyalkylene glycol and a monohydric or polyhydric alcohol, polyoxyalkylene sugar ethers, condensates formed between a fatty amine and a polyoxyalkylene glycol, alkyl or alkenyl polyglycosides, and the like.

Among these nonionic surfactants, a nonionic surfactant being highly safe and showing a liquid state or a pasty state at 25° C. is desirable, and one having a polyglycerol moiety having an average degree of polymerization of from 2 to 30 or a polyoxyalkylene group having an alkylene moiety with 2 to 4 carbon atoms, and an average number of moles added of from 1 to 80 is preferable.

Therefore, specific examples of preferred nonionic surfactants include polyglycerol(average degree of polymerization: 3 to 15) fatty acid(number of carbon atoms: 12 to 24) esters, polyoxyethylene(average number of moles added: 3 to 40) alkyl(number of carbon atoms: 12 to 24) ethers, polyoxyethylene(average number of moles added: 3 to 40) fatty acid(number of carbon atoms: 12 to 24) esters, polyoxyethylene(average number of moles added: 3 to 40) glycerol fatty acid(number of carbon atoms: 12 to 24) esters, polyoxyethylene(average number of moles added: 3 to 40) hardened castor oils, polyoxyethylene(average number of moles added: 3 to 40) alkyl ether polyglycosides, fatty acid(number of carbon atoms: 12 to 24) polyoxyethylene(average number of moles added: 3 to 40) sorbitan esters, and the like.

The nonionic surfactant may be used alone or as a mixture of two or more kinds thereof having different HLBs or the like.

The content of the nonionic surfactant, for instance, in the case where the nonionic surfactant is used for cleansing cosmetics, is preferably from 0.1 to 80% by weight, more preferably from 1 to 40% by weight, especially preferably from 2 to 30% by weight, of the polyglycerol medium-chain fatty acid ester-containing composition, from the viewpoint of remover capability and rinsability.

The polyglycerol medium-chain fatty acid ester-containing composition of the present invention may further comprise an oil agent. It is preferable that the oil agent shows a liquid or pasty state at 25° C. In the present invention, the phrase “shows(showing) a liquid or pasty state at 25° C.” refers to one having a melting point of 25° C. or lower, or one having fluidity in which solid substances are dispersed in a liquid substance having a melting point of less than 25° C.

The oil agent includes natural animal or plant fats and oils and semi-synthesized fats and oils, hydrocarbon oils, ester oils, glyceride oils, silicone oils, fat-soluble vitamins, higher fatty acids, components of purified oils from animals and plants or synthetic oils, and the like.

The natural animal or plant fats and oils and semi-synthesized fats and oils include avocado oil, linseed oil, almond oil, olive oil, wheat germ oil, sesame oil, rice germ oil, rice bran oil, safflower oil, soybean oil, evening primrose oil, Indian corn oil, rapeseed oil, horse fat, palm oil, palm kernel oil, castor oil, sunflower oil, jojoba oil, macadamia nut oil, coconut oil, hardened coconut oil, peanut oil, lanolin and the like.

The hydrocarbon oil includes squalane, squalene, liquid paraffin, Vaseline and the like.

The ester oil includes diisobutyl adipate, 2-hexyldecyl adipate, di-2-heptylundecyl adipate, isostearyl isostearate, trimethylolpropane triisostearate, cetyl 2-ethylhexanoate, neopentyl glycol di-2-ethylhexanoate, trimethylolpropane tri-2-ethylhexanoate, pentaerythritol tetra-2-ethylhexanoate, cetyl octanoate, oleyl oleate, octyl dodecyl oleate, decyl oleate, neopentyl glycol dicaprate, 2-ethylhexyl succinate, isocetyl stearate, butyl stearate, diisopropyl sebacate, cetyl lactate, tetradecyl lactate, isopropyl myristate, octyl palmitate, 2-ethylhexyl palmitate, 2-hexyldecyl palmitate, 2-heptylundecyl palmitate, cholesteryl 12-hydroxystearate, phytostearyl oleate, diisostearyl malate, paramethoxycinnamic acid ester, pentaerythritol tetrarosinate, and the like.

The glyceride oil includes glyceryl triisostearate, glyceryl triisopalmitate, glyceryl tri-2-ethylhexanoate, glycerol tritetradecanoate, glyceryl diparamethoxycinnamate monoisoocylate, and the like.

The silicone oil includes higher alkoxy-modified silicones, alkyl-modified silicones and higher fatty acid ester-modified silicones such as dimethyl polysiloxane, methylphenyl polysiloxane, methyl hydrogen polysiloxane, octamethyl cyclopentanesiloxane, decamethyl cyclohexasiloxane, and stearoxy silicone; and the like.

The fat-soluble vitamin includes tocopherol or a derivative thereof, retinol or a derivative thereof, and the like.

The content of the oil agent, in the case, for instance, where the oil agent is used for cleansing cosmetics, preferably from 10 to 99.8% by weight, more preferably from 50 to 99% by weight, especially preferably from 70 to 98% by weight, of the polyglycerol medium-chain fatty acid ester-containing composition, from the viewpoint of remover capability and rinsability.

Since the polyglycerol medium-chain fatty acid ester-containing composition of the present invention can form a microemulsion capable of solubilizing a large amount of water, the composition may be not only a non-aqueous composition which contains no water but also an aqueous composition containing water.

The content of water is preferably 80% by weight or less, more preferably from 0.01 to 80% by weight, even more preferably from 1 to 50% by weight, of the polyglycerol medium-chain fatty acid ester-containing composition. In addition, in this case, the weight ratio of the polyglycerol medium-chain fatty acid ester to the oil agent (polyglycerol medium-chain fatty acid ester/oil agent) is preferably from 10/0.1 to 0.1/10, from the viewpoint of preventing phase separation by containment of water.

The polyglycerol medium-chain fatty acid ester-containing composition of the present invention may further contain an oily gelation agent, a lower alcohol, a powder, functional beads, a capsule, an antioxidant, an ultraviolet absorbent, a plant extract, a moisturizing agent, a bactericidal agent, an anti-inflammatory agent, a preservative, a pigment, a perfume or the like.

The oily gelation agent includes organic modified bentonite, hydrophobic silicic acid, silicic acid anhydride, starch fatty acid esters, and the like. The oily gelation agent is useful in the adjustment of viscosity. For instance, the properties of the composition of the present invention can be properly adjusted to a state of transparent to semi-transparent liquid to viscous paste.

The polyglycerol middle-chain fatty acid ester-containing composition of the present invention can form a water-in-oil type microemulsion capable of solubilizing a large amount of water. Also, in the process of adding water, since the interfacial tension between oil and water phases is reduced by the formation of the microemulsion, the microemulsion is easily emulsified by further adding a large amount of water to form fine emulsion, the composition also has excellent water dispersibility and self-emulsifiability in water. Furthermore, since the polyglycerol is prepared by using glycerol as a starting raw material, the resulting composition has low skin irritation, and also excellent properties in the aspect of safety. Therefore, the composition can be used for various manufactured articles such as cosmetics and toiletry manufactured articles such as cleaning agent for scalp before shampooing.

Therefore, as more preferable one embodiment using the polyglycerol medium-chain fatty acid ester-containing composition of the present invention, there are further provided cosmetics comprising the composition of the present invention. The cosmetics include cleansing cosmetics, bathing cosmetics, skin care cosmetics such as emulsion cosmetics, and the like. Especially, in the cleansing cosmetics and the bathing cosmetics, the effects by the composition of the present invention are more remarkably exhibited.

Specifically, the cleansing cosmetics comprising the polyglycerol medium-chain fatty acid ester-containing composition of the present invention have excellent affinity to makeup soil, and have remarkably excellent remover capability as compared to those of conventional oil-based makeup removers in the cleansing step, without losing their remover capability even in a wet skin state. Also, the bathing cosmetics of the present invention have excellent dispersibility and self-emulsifiability in hot water bath, so that even higher bathing effects are exhibited.

The content of the polyglycerol medium-chain fatty acid ester-containing composition in the cosmetics of the present invention can be properly selected depending upon the manufactured article used and their purposes, and is not particular limited. For instance, in the case where the composition is used for cleansing cosmetics, it is preferable that the content of the polyglycerol medium-chain fatty acid ester-containing composition is adjusted so that the content of the polyglycerol medium-chain fatty acid ester is preferably from 0.1 to 80% by weight, more preferably from 1 to 50% by weight, even more preferably from 2 to 30% by weight, of the cosmetics, from the viewpoint of affinity to makeup soil.

The cosmetics of the present invention can be prepared in the same manner as usual depending upon their purposes and the like except that the polyglycerol medium-chain fatty acid ester-containing composition of the present invention is used. The time of adding or method or adding the polyglycerol medium-chain fatty acid ester-containing composition is not particularly limited as long as the exhibition of the desired effects of the present invention is obtained.

### EXAMPLES

The present invention will be specifically described hereinbelow by the Examples, without intending to limit the scope of the present invention thereto.

### Examples 1 to 9 and Comparative Examples 1 to 5

An oil-based makeup remover comprising a polyglycerol medium-chain fatty acid ester- containing composition was prepared by a conventional method using the raw materials shown in Table 1.

1. Evaluation for Feel of Use

Ten specialist panelists who were provided with makeup took on their hands 1 g of a makeup remover, and applied over an entire face with both hands and massaged for 30 seconds, and thereafter washed off with water. In the course of treatment, the affinity of makeup soil, rinsability and oily feel after washing were evaluated in 5 ranks from 1 (poor or being sensed) to 5 (excellent or not being sensed), and an average score of 10 individuals was calculated. The results are shown in Table 1 in accordance with the following evaluation criteria.

[Evaluation Criteria]

◯: An average score of 4 or more;

Δ: An average score of 3 or more and less than 4; and

×: An average score of less than 3.

2. Evaluation for Remover Capability (State Where Skin Was Not Being in Wet State)

A lipstick was applied to forearm in an area of 2 cm×2 cm, and allowed to stand for 30 minutes. Thereafter, 0.5 of a makeup remover was applied thereto. Thereafter, the forearm was massaged for 30 seconds, so as to remove the lipstick therefrom, and washed off with water. The extent of removal of the lipstick was visually examined. The results are shown in Table 1 in accordance with the following evaluation criteria.

[Evaluation Criteria]

◯: Excellently removed;

Δ: Not removed much; and

×: Not removed at all.

3. Evaluation for Remover Capability (State Where Skin Was Being in Wet State)

A lipstick was applied to forearm in an area of 2 cm×2 cm, and allowed to stand for 30 minutes. The portion to which the lipstick was applied was wetted with water, and 0.5 of a makeup remover was applied thereto. Thereafter, the forearm was massaged for 30 seconds, so as to remove the lipstick therefrom, and washed off with water. The extent of removal of the lipstick was visually examined. The results are shown in Table 1 in accordance with the following evaluation criteria.

[Evaluation Criteria]

◯: Excellently removed;

Δ: Not removed much; and

×: Not removed at all.

4. Confirmation for Formation of Microemulsion

One gram of a makeup remover was weighed in a test tube, and 1 g of water was added thereto while stirring. The state was visually examined. In the case where a microemulsion was formed, the solution becomes transparent. The results are shown in Table 1 in accordance with the following evaluation criteria.

[Evaluation Criteria]

◯: Transparent;

Δ: Semi-transparent; and

×: White turbid.

TABLE 1

| | | | | | | | | | | Comparative | | | | |
|---|---|---|---|---|---|---|---|---|---|---|---|---|---|---|
| | Examples | | | | | | | | | Examples | | | | |
| | 1 | 2 | 3 | 4 | 5 | 6 | 7 | 8 | 9 | 1 | 2 | 3 | 4 | 5 |
| Polyglycerol Medium-Chain Fatty Acid Ester | | | | | | | | | | | | | | |
| Pentadecaglycerol Pentacaprylate (HLB: 12) | | 10 | | 15 | 10 | | 5 | 10 | 10 | | | | | |
| Eicosaglycerol Hexacaprylate (HLB: 13) | 10 | | 10 | | | 10 | 5 | | | | | | | 20 |
| Nonionic Surfactant | | | | | | | | | | | | | | |
| Decaglycerol Monoisostearate | 20 | | | | | | | | | 20 | | | | |
| Decaglycerol Trilaurate | | 20 | | | | | | | 20 | | | | | |
| Decaglycerol Diisostearate | | | 10 | | | | 10 | | | | | 10 | | |
| Polyoxyethylene(10) Hardened Castor Oil* | | | | 15 | | | 10 | | | | 20 | 10 | | |
| Polyoxyethylene(20) Sorbitan Monooleate* | | | | | 20 | | | 5 | | | | | 20 | |
| Polyoxyethylene(10) Oleyl Ether* | | | | | | 10 | | 5 | | | | | | |
| Oil Agent | | | | | | | | | | | | | | |
| Octyl Palmitate | 50 | 50 | | 70 | 30 | 60 | 50 | | 20 | 60 | 80 | | 40 | |
| Liquid Paraffin | 10 | 10 | 10 | | 5 | 10 | | 20 | | 10 | | 10 | 5 | 10 |
| Isopropyl Myristate | | | 70 | | 30 | | 10 | 30 | 10 | | | 70 | 30 | 70 |
| Olive Oil | 10 | 10 | | | 5 | 10 | | 30 | | 10 | | | 5 | |
| Water | | | | | | | | | 40 | | | | | |
| Feel of Use | | | | | | | | | | | | | | |
| Affinity to Soil | ◯ | ◯ | ◯ | ◯ | ◯ | ◯ | ◯ | ◯ | ◯ | Δ | ◯ | ◯ | ◯ | X |
| Rinsability | ◯ | ◯ | ◯ | ◯ | ◯ | ◯ | ◯ | ◯ | ◯ | Δ | ◯ | ◯ | ◯ | X |
| Absence of Oily Feel | ◯ | ◯ | ◯ | ◯ | ◯ | ◯ | ◯ | ◯ | ◯ | Δ | ◯ | Δ | Δ | X |
| Remover Capability | | | | | | | | | | | | | | |
| Skin not being in wet state | ◯ | ◯ | ◯ | ◯ | ◯ | ◯ | ◯ | ◯ | ◯ | Δ | ◯ | ◯ | Δ | X |
| Skin being in wet state | ◯ | ◯ | ◯ | ◯ | ◯ | ◯ | ◯ | ◯ | ◯ | X | X | X | X | X |
| Formation of Microemulsion | ◯ | ◯ | ◯ | ◯ | ◯ | ◯ | ◯ | ◯ | ◯ | X | X | X | X | X |

Note)
The used amount is expressed by parts by weight.
*Number inside parenthesis shows an average number of moles of oxyethylene group added.

It can be seen from the above results that all of the oil-based makeup removers of the Examples in which the microemulsion was formed had excellent feel of use, and had excellent remover capability not only in the case where the skin was not in a wet state but also in a wet state, as compared to those of Comparative Examples.

### INDUSTRIAL APPLICABILITY

The polyglycerol medium-chain fatty acid ester-containing composition of the present invention can be suitably used for cosmetics such as cleansing cosmetics and bathing cosmetics.

1. A polyglycerol medium-chain fatty acid ester-containing composition comprising a polyglycerol medium-chain fatty acid ester formed by esterification of a medium-chain fatty acid having 6 to 10 carbon atoms and a polyglycerol having an average degree of polymerization of 3 or more and less than 100, and a nonionic surfactant.

2. The polyglycerol medium-chain fatty acid ester-containing composition according to claim 1, further comprising an oil agent showing a liquid or pasty state at 25° C.

3. The polyglycerol medium-chain fatty acid ester-containing composition according to claim 1 or 2, further comprising from 0 to 80% by weight of water.

4. Cosmetics comprising the polyglycerol medium-chain fatty acid ester-containing composition as defined in claim 1.

5. The cosmetics according to claim 4, wherein the cosmetics are cleansing cosmetics or bathing cosmetics.
